(19) United States
(12) Patent Application Publication
(10) Pub. No.: US 2011/0178106 A1
(43) Pub. Date: Jul. 21, 2011

(54) 5-QUINOLINONE AND IMIDAZOPYRIDINE COMPOUNDS AND USE THEREOF

(75) Inventors: Joseph A. Maddry, Birmingham, AL (US); Adam Keeton, Birmingham, AL (US); Jason Whitt, Birmingham, AL (US); Gary Piazza, Birmingham, AL (US)

(73) Assignee: SOUTHERN RESEARCH INSTITUTE OFFICE OF COMMERCIALIZ, BIRMINGHAM, AL (US)

(21) Appl. No.: 13/002,942

(22) PCT Filed: Jul. 10, 2009
(86) PCT No.: PCT/US09/50239

§ 371 Date: Apr. 4, 2011

Related U.S. Application Data
(60) Provisional application No. 61/079,604, filed on Jul. 10, 2008.

(51) Int. Cl. A61K 31/519; C07D 401/04; C07D 405/04; C07D 487/04; A61K 31/4709; A61K 31/47; C12Q 1/02; A61P 35/00; A61P 29/00; A61P 31/00; A61P 33/00; A61P 31/10

(52) U.S. Cl. 514/259.1; 546/159; 544/281; 514/313; 435/29

(57) ABSTRACT

5-Quinolinone and Imidazopyrimidine compounds are provided that are useful for inhibiting the efflux of any therapeutic agent that is a MRP1 substrate. Also provided is a method for screening to identify additional MRP1 inhibitors.

### FEDERALLY SPONSORED RESEARCH AND DEVELOPMENT

This invention was partially supported by a grant No. 5 U54HG003917 from National Institute of Health and the US Government has certain rights in the invention.

### TECHNICAL FIELD

The present disclosure relates to certain 5-quinolinone and imidazopyrimidine compounds. The 5-quinolinone and imidazopyrimidine of the present disclosure are especially suitable for enhancing the efficacy of various pharmacological agents. Compounds of the present disclosure are particularly useful as multi-drug resistant protein 1 (MRP1) inhibitors.

### BACKGROUND OF DISCLOSURE

Drug resistance, whether intrinsic or acquired, is a major clinical obstacle, which limits the efficacy of a number of treatments including cancer chemotherapy. With respect to cancer chemotherapy, multi-drug resistance (MDR) is a phenomenon by which tumor cells display or develop resistance to a number of structurally and functionally different anticancer drugs. A significant factor that contributes to MDR is the over-expression of certain membrane transport proteins that pump chemotherapeutic drugs out of the cell to reduce the intracellular concentration and limit the clinical effectiveness of chemotherapy. The most well characterized transport proteins responsible for MDR are the P-glycoprotein (P-gp) and the multi-drug resistance protein (MRP) family of transporters.

The ATP-binding cassette (ABC) class of membrane transporters represents a large family of approximately 50 different proteins that are highly conserved and display similar function across prokaryotic and eukaryotic organisms. Several ABC family members have been shown to be over expressed in tumors and implicated in multidrug resistance. MRP-1 is one such transport protein that is often over expressed in carcinomas.

Inhibition of membrane transporters is an attractive therapeutic strategy to enhance chemotherapy efficacy. P-gp has been well studied for the past two decades and a number of inhibitors have been evaluated in clinical trials. However, there have been no P-gp inhibitors approved by the FDA, which is largely attributed to excessive toxicity when combined with chemotherapy and caused by interfering with the pharmacokinetics of chemotherapy. Less is known regarding the binding and transport properties of MRP1. Recent studies, however suggest that it may be feasible to develop novel MRP1 inhibitors that reverse MDR without interfering with pharmacokinetics of chemotherapy. Unfortunately, few MRP1 selective inhibitors are known that display desired potency and selectivity.

### SUMMARY OF DISCLOSURE

The present disclosure is concerned with compounds represented by the formulae:

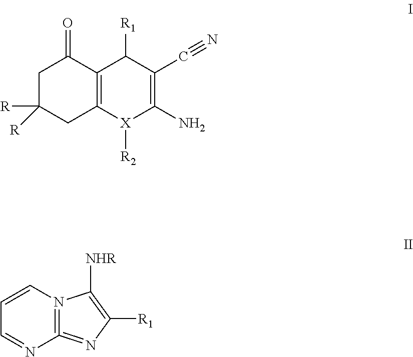

and pharmaceutically acceptable salts thereof; solvates thereof, and prodrugs thereof.

In formula I, X is N or O; each R is individually selected from the group consisting of H and alkyl; R₁ is a substituted- or unsubstituted-aryl or substituted- or unsubstituted-heteroaryl; and R₂ is phenyl or substituted phenyl.

In formula II, R is selected from the group consisting of alkyl, cycloalkyl, substituted- or unsubstituted-aryl or substituted- or unsubstituted-heteroaryl, and adamantyl; and R₂ is substituted- or unsubstituted-aryl or substituted- or unsubstituted-heteroaryl.

The present disclosure is also concerned with pharmaceutical compositions comprising an effective amount of a compound or pharmaceutically acceptable salt thereof, or a solvate thereof, or prodrug thereof as disclosed above and a pharmaceutically acceptable carrier.

Another aspect of the present disclosure relates to a method for enhancing the efficacy or reducing the toxicity of a pharmacological agent which comprises administering to a patient in need thereof, an effective amount of a compound or pharmaceutically acceptable salt thereof, or a solvate thereof, or prodrug thereof as disclosed above.

A still further aspect of the present disclosure is concerned with a method for screening for compounds for use as MRP inhibitors which comprises exposing a sample compound to MRP1 over expressing human small cell lung tumor line and measuring the sensitivity of the cell both in the absence and the presence of a subtoxic concentration of a pharmaceutical agent.

Still other objects and advantages of the present disclosure will become readily apparent by those skilled in the art from the following detailed description, wherein it is shown and described preferred embodiments, simply by way of illustration of the best mode contemplated. As will be realized the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF FIGURES

FIGS. 1A-1D illustrate the selectivity of SRI 22029 to inhibit MRP1 mediated drug resistance.

FIGS. 2A-2D illustrate the selectivity of SRI 22156 to inhibit MRP1 mediated drug resistance.

### BEST AND VARIOUS MODES FOR CARRYING OUT DISCLOSURE

The present disclosure is concerned with 5-quinolinone and imidazopyrimidine compounds represented by the formulae I and II, respectively:

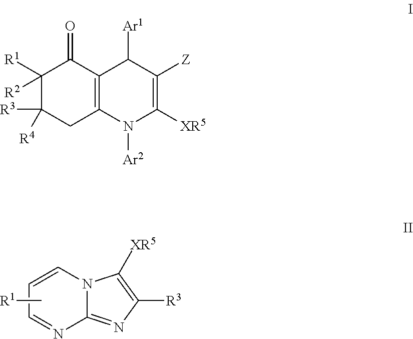

In the first structure, R¹-R⁶ are independently hydrogen, saturated or unsaturated alkyl, cycloalkyl, aryl, or heterocyclic; Ar¹-Ar² are independently aryl, heteroaryl, or cycloalkyl; X=O or N—R⁶; and Z=an electron withdrawing substituent such as cyano, azido, or halogen.

In the second structure, R¹ represents one, two, or three independent substituents of hydrogen, alkyl or unsaturated alkyl, cycloalkyl, aryl, or heterocyclic moiety, or halogen; R²-R⁴ are independently hydrogen, alkyl or unsaturated alkyl, cycloalkyl, aryl, or heterocyclic moiety; and X=O or N—R⁴.

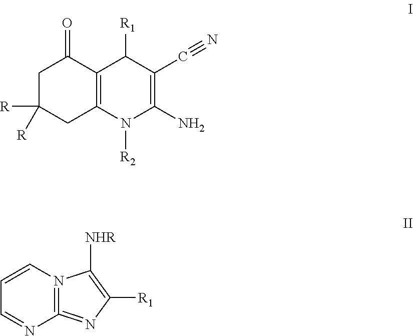

and pharmaceutically acceptable salt thereof, solvates thereof, and prodrugs thereof.

In formula I, X is N or O; each R is individually selected from the group consisting of H and alkyl; R₁ is a substituted- or unsubstituted-aryl or substituted- or unsubstituted-heteroaryl; and R₂ is phenyl or substituted phenyl.

In formula II, R is selected from the group consisting of alkyl, cycloalkyl, substituted- or unsubstituted-aryl or substituted- or unsubstituted-heteroaryl, and adamantyl; and R₂ is substituted- or unsubstituted-aryl or substituted- or unsubstituted-heteroaryl.

Listed below are definitions of various terms used to describe this disclosure. These definitions apply to the terms as they are used throughout this specification, unless otherwise limited in specific instances, either individually or as part of a larger group. Also, in the formulae described and claimed herein, it is intended that when any symbol appears more than once in a particular formula or substituent, its meaning in each instance is independent of the other.

“Effective amount” refers to an amount of a compound as described herein that may be therapeutically effective to enhance the efficacy of a pharmacological agent. The precise amount of these compounds required will vary with the particular compounds or derivatives employed, the age and condition of the subject to be treated, and the nature and severity of the condition. However, the effective amount may be determined by one of ordinary skill in the art once aware of this disclosure without undue experimentation.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. Typical inorganic acids used to form such salts include hydrochloric, hydrobromic, hydroiodic, nitric, sulfuric, phosphoric, hypophosphoric and the like. Salts derived from organic acids, such as aliphatic mono and dicarboxylic acids, phenyl substituted alkonic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, β-hydroxybutyrate, butyne-1,4-dioate, hexyne-1,4-dioate, cabrate, caprylate, chloride, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toleunesulfonate, xylenesulfonate, tartarate, and the like.

Bases commonly used for formation of salts include ammonium hydroxide and alkali and alkaline earth metal hydroxides, carbonates, as well as aliphatic and primary, secondary and tertiary amines, aliphatic diamines. Bases especially useful in the preparation of addition salts include sodium hydroxide, potassium hydroxide, ammonium hydroxide, potassium carbonate, methylamine, diethylamine, and ethylene diamine.

A “Prodrug” is a compound that is converted within the body into its active form that has a medical effect. Prodrugs may be useful when the active drug may be too toxic to administer systemically, the active drug is absorbed poorly by the digestive tract, or the body breaks down the active drug before it reaches its target. Methods of making prodrugs are disclosed in Hans Bundgaard, DESIGN OF PRODRUGS (Elsevier Science Publishers B.V. 1985), which is incorporated herein by reference in its entirety.

Prodrug forms of the compounds bearing various nitrogen functions (amino, hydroxyamino, hydrazino, guanidino, amidino, amide, etc.) may include the following types of derivatives where each R group individually may be hydrogen, substituted or unsubstituted alkyl, aryl, alkenyl, alkynyl, heterocycle, alkylaryl, aralkyl, aralkenyl, aralkynyl, cycloalkyl or cycloalkenyl groups as defined above.

Carboxamides, —NHC(O)R

Carbamates, —NHC(O)OR

(Acyloxy)alkyl Carbamates, NHC(O)OROC(O)R

Enamines, —NHCR(═CHCRO₂R) or —NHCR(═CHCRONR₂)

Schiff Bases, —N═CR₂

Mannich Bases (from carboximide compounds), RCONHCH₂NR₂

Preparations of such prodrug derivatives are discussed in various literature sources (examples are: Alexander et al., J. Med. Chem. 1988, 31, 318; Aligas-Martin et al., PCT WO pp/41531, p. 30). The nitrogen function converted in preparing these derivatives is one (or more) of the nitrogen atoms of a compound of the invention.

Prodrug forms of carboxyl-bearing compounds of the disclosure include esters (—CO₂R) where the R group corresponds to any alcohol whose release in the body through enzymatic or hydrolytic processes would be at pharmaceutically acceptable levels.

Another prodrug derived from a carboxylic acid form of the disclosure may be a quaternary salt type

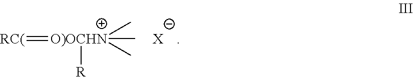

of structure described by Bodor et al., J. Med. Chem. 1980, 23, 469.

It is of course understood that the compounds of the present disclosure relate to all optical isomers and stereo-isomers at the various possible atoms of the molecule.

“Solvates” refers to the compound formed by the interaction of a solvent and a solute and includes hydrates. Solvates are usually crystalline solid adducts containing solvent molecules within the crystal structure, in either stoichiometric or non-stoichiometric proportions.

The term “halogen” or “halo” refers to fluorine, chlorine, bromine and iodine.

The term “aryl” refers to monocyclic or bicyclic aromatic hydrocarbon groups having 6 to 12 carbon atoms in the ring portion, such as phenyl, naphthyl, biphenyl and diphenyl groups, each of which may be substituted. The aromatic or aryl groups are more typically phenyl and alkyl substituted aromatic groups (aralkyl) such as phenyl C₁₋₃ alkyl and benzyl.

The term “aralkyl” or “alkylaryl” or “alaryl” refers to an aryl group bonded directly through an alkyl group, such as benzyl or phenethyl.

The term “substituted aryl” or “substituted alkylaryl” refers to an aryl group or alkylaryl group substituted by, for example, one to four substituents such as alkyl; substituted alkyl, halo and alkoxy. “Substituted benzyl” refers to a benzyl group substituted by, for example, any of the groups listed above for substituted aryl.

The term “cycloalkyl” refers to optionally substituted, saturated cyclic hydrocarbon ring systems, preferably containing 1 to 3 rings and 3 to 7 carbons per ring which may be further fused with an unsaturated C₃-C₇ carbocyclic ring. Exemplary groups include cyclopropyl, cyclobutyl, cyclopentyl, cyclohexyl, cycloheptyl, cyclooctyl, cyclodecyl, cyclododecyl and adamantyl. Exemplary substituents include one or more alkyl groups as described above, or one or more groups described above as alkyl substituents.

The term “alkyl” refers to straight or branched chain unsubstituted hydrocarbon groups of 1 to 20 carbon atoms, and more typically 1 to 8 carbon atoms and even more typically unsubstituted alkyl groups of 1 to 4 carbon atoms. Examples of suitable alkyl groups include methyl, ethyl and propyl. Examples of branched alkyl groups include isopropyl and t-butyl. Examples of unsaturated alkyl groups include ethynyl, cyclopentenyl, and allyl.

The term “heteroaryl”, refer to an optionally substituted, unsaturated aromatic cyclic group, for example, which is a 4 to 7 membered monocyclic, 7 to 11 membered bicyclic, or 10 to 15 membered tricyclic ring system, which has at least one heteroatom and at least one carbon atom in the ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized and the nitrogen heteroatoms may also optionally be quaternized. The heterocyclic group may be attached at any heteroatom or carbon atom. Examples of heteroaryls include, but are not limited to pyrrole, imidazole, pyrazole, pyridine, pyrazine, pyrimidine, pyridazine, indolizine, isoindole, indole, indazole, purine, quinoxaline, quinazoline, cinnoline, thiophene, furan and isopyrrole. The heteroaromatic moieties can be optionally substituted as described above for aryl, including substituted with one or more substituents selected from alkoxy, halo, and alkyl.

The terms “heterocycle”, “heterocyclic” and “heterocyclo” refer to an optionally substituted, fully saturated or unsaturated, aromatic or nonaromatic cyclic group, for example, which is a 4 to 7 membered monocyclic, 7 to 11 membered bicyclic, or 10 to 15 membered tricyclic ring system, which has at least one heteroatom and at least one carbon atom in the ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized and the nitrogen heteroatoms may also optionally be quaternized. The heterocyclic group may be attached at any heteroatom or carbon atom. Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, isopyrrole, 1,2,3-triazole, 1,2,4-triazole, oxazole, thiazole, pyrimidine, aziridines, thiazole, 1,2,3-oxadiazole, thiazine, pyrrolidine, oxaziranes, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N6-alkylpurines, N6-benzylpurine, N6-halopurine, N6-vinypurine, N6-acetylenic purine, N6-acyl purine, N6-hydroxyalkyl purine, N6-thioalkyl purine, thymine, cytosine, 6-azapyrimidine, 2-mercaptopyrmidine, uracil, N5-alkyl-pyrimidines, N5-benzylpyrimidines, N5-halopyrimidines, N5-vinyl-pyrimidine, N5-acetylenic pyrimidine, N5-acyl pyrimidine, N5-hydroxyalkyl purine, and N6-thioalkyl purine, and isoxazolyl. The heteroaromatic and heterocyclic moieties can be optionally substituted as described above for aryl, including substituted with one or more substituents selected from hydroxyl, amino, alkylamino, acylamino, alkoxy, aryloxy, alkyl, heterocycle, halo, carboxy, acyl, acyloxy, amido, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

Compounds accordingly to the present disclosure are especially suitable for enhancing the efficacy of various pharmacological agents and are particularly useful for inhibiting the efflux of any therapeutic agent that is a MRP1 substrate. For example, compounds accordingly to the present disclosure will enhance the anticancer efficacy of such cancer chemotherapeutic drugs in mammals as doxorubicin, epirubicin, idarubicin, daunomycin or other anthracyclines. Additionally, compounds accordingly to the present disclosure should enhance the activity of vincristine, vinorelbine, and other vinca alkaloids as well as other drug classes that are known to be substrates for MRP1 including methotrexate and other drugs including antifolates, etoposide, menograril, colchicines, VP-16, gramicidin as well as various non-steroidal anti-inflammatory drugs. Moreover, compounds accordingly to the present disclosure will enhance activity of antibiotics as well as certain other antiparasitic and antifungal drugs in mammals.

Compounds accordingly to the present disclosure can be administered either prior to treatment with chemotherapy or other drug therapy, during treatment, or post-treatment.

Compounds accordingly to the present disclosure were identified by a novel cell-based assay to identify MRP1 inhibitors using high throughput screening. The assay involves a MRP1 over expressing human small cell tumor line, such as H69AR and measuring the sensitivity of cells in the absence and presence of a subtoxic concentration of a known MRP1 substrate and in particular doxorubicin (IC20 value). Sensitivity was measured using a standard cell viability readout, which in this case involved measuring ATP levels by a commercial assay. The unique design of the assay allowed for readily differentiating cytotoxic compounds that were of no interest, from compounds that could selectively enhance the cytotoxicity of the MRP1 substrate doxorubicin in the drug resistant tumor cells. Additional experiments were performed in H69AR cells to determine the ability of a subtoxic concentration of the tested compounds to shift the doxorubicin dose-response curve to the left (i.e., decrease the IC50 value), which is indicative of activity to inhibit efflux. Using the dose-shift assay as described above, compounds were identified as active based on their ability to enhance the sensitivity of MRP1 over expressing drug resistant cells to doxorubicin. The results for analogs with the 5-quinolinone and imidazopyrimidine are summarized in Tables 1 and 2, respectively.

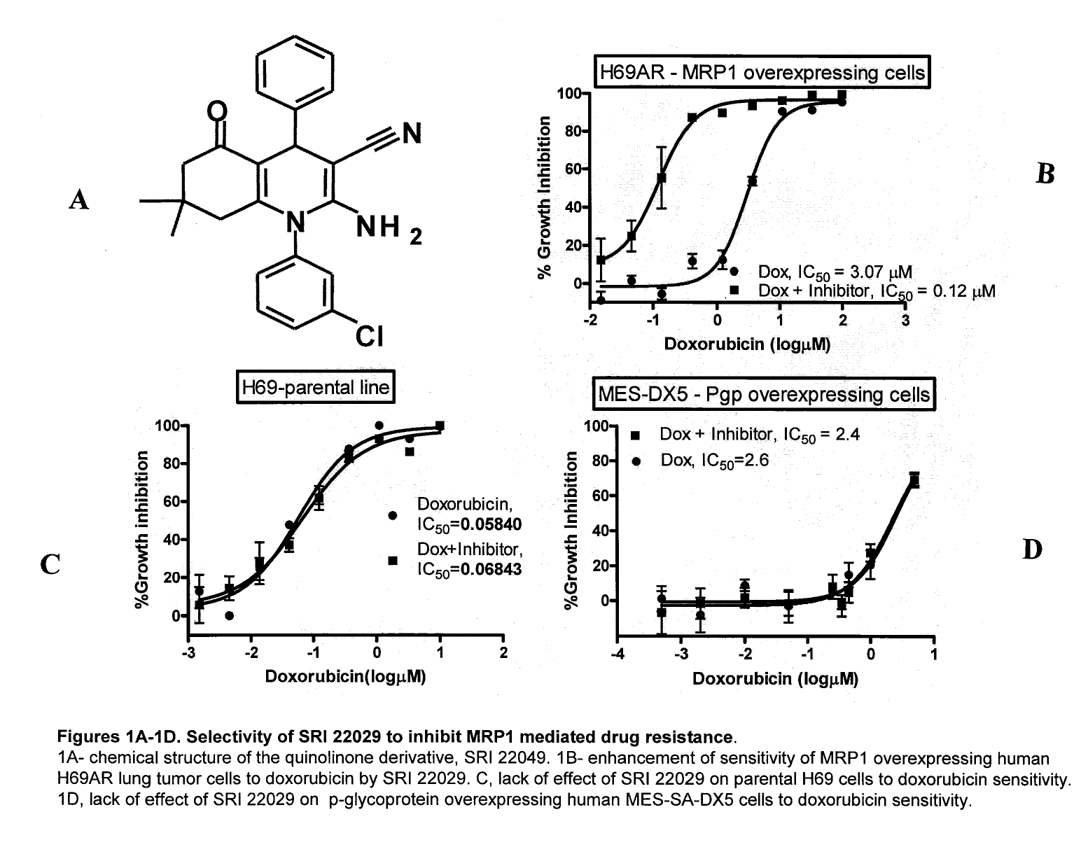

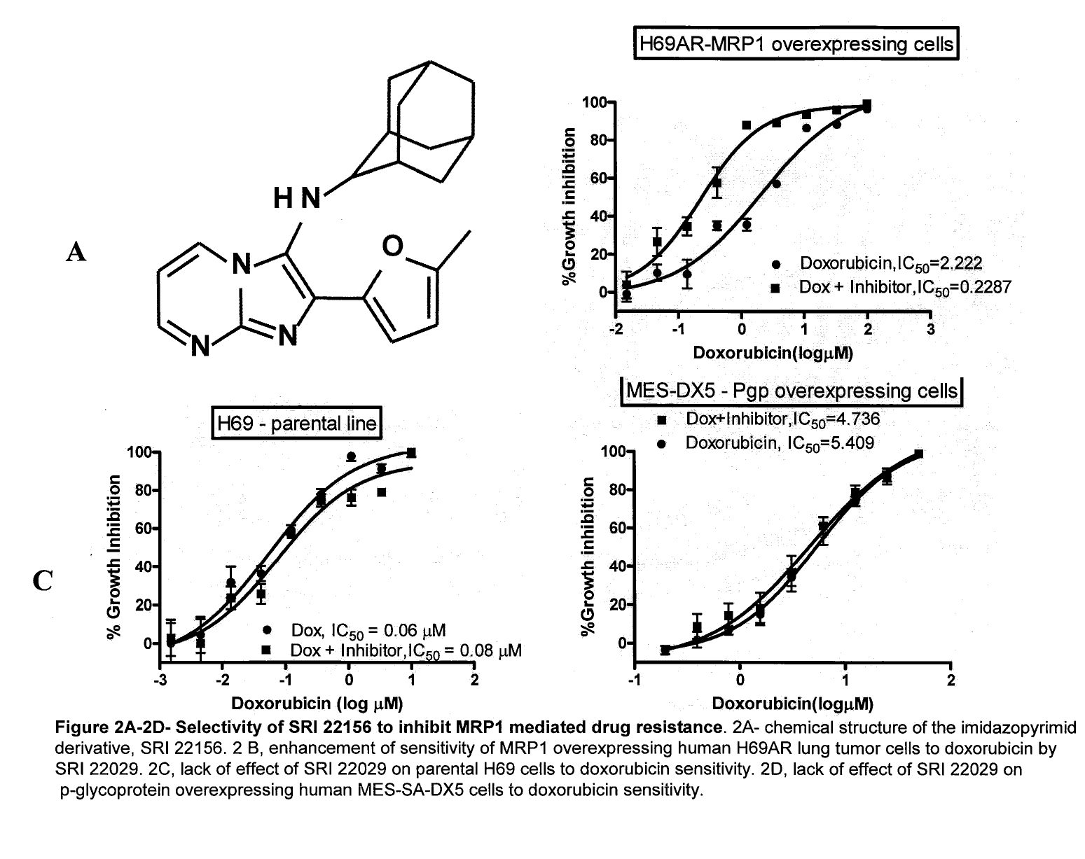

Additional studies were also conducted with the most active compounds to assess selectivity. By comparing with the H69AR parental line, H69, and a P-gp over expressing line, MES-SA-DX5, both series of compounds were shown to be highly selective for MRP1 over expressing cells. Along these lines, please see FIGS. 1 and 2. In particular, FIG. 1 illustrates selectivity of the compound identified as SRI 22029 to inhibit MRP1 mediated drug resistance. FIG. 1A illustrates a chemical structure of the quinolinone derivative, SRI 22049. FIG. 1B illustrates enhancement of sensitivity of MRP1 overexpressing human H69AR lung tumor cells to doxorubicin by SRI 22029. FIG. 1C illustrates lack of effect of SRI 22029 on parental H69 cells to doxorubicin sensitivity. FIG. 1D illustrates lack of effect of SRI 22029 on p-glycoprotein overexpressing human MES-SA-DX5 cells to doxorubicin sensitivity. FIG. 2 illustrates selectivity of the compound identified as SRI 22156 to inhibit MRP1 mediated drug resistance. FIG. 2A illustrates chemical structure of the imidazopyrimidine derivative, SRI 22156. FIG. 2B illustrates enhancement of sensitivity of MRP1 overexpressing human H69AR lung tumor cells to doxorubicin by SRI 22029. FIG. 2C illustrates lack of effect of SRI 22029 on parental H69 cells to doxorubicin sensitivity. FIG. 2D illustrates lack of effect of SRI 22029 on p-glycoprotein overexpressing human MES-SA-DX5 cells to doxorubicin sensitivity. In addition, profiling studies were conducted to evaluate the compounds for ability to inhibit 250 known enzymes. Both classes of compounds were found to lack significant inhibitory activity on other enzymes, which may be indicative of a high degree of selectivity for MRP1.

The compounds tested that show a “left fold shift” preferably a 2 fold or greater “left fold shift” and more preferably a 10 fold or greater “left fold shift” in the IC50 value to doxorubicin are predicted to have utility for inhibiting the efflux of any therapeutic agent that is a MRP1 substrate.

The inhibitors are expected to be active in mammals if administered PO, IV or IP at a dosage of approximately 10-1000 mg/kg body weight in a pharmaceutically acceptable formulation. The inhibitors are expected to be active when administered either prior to, during, or following the treatment with the pharmacological agent. When administered either prior to or following the treatment with the pharmacological agent, the inhibitor is typically administered within about 24 hours of the treatment.

Representative compounds suitable for the treatments according to the present disclosure along with their left shift values are disclosed in the following Tables 1 and 2:

TABLE 1

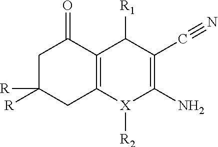

| SRI # | X | R | R1 | R2 | Fold Left Shift |
|---|---|---|---|---|---|
| 22061 | N | H | 4-Pyridine | 4-Methoxyphenyl | 0 |
| 22054 | N | H | 2-Furan | 4-methoxyphenyl | 2.13 |
| 22030 | N | Methyl | Phenyl | 3,4-Dichlorophenyl | 15.07 |
| 22062 | N | H | 4-Pyridine | 4-Methoxyphenyl | 3.50 |
| 22059 | N | Methyl | 2-Furan | 4-methoxyphenyl | 3.82 |
| 22053 | N | H | 3-Pyridine | 4-Methoxyphenyl | 3.79 |
| 22029 | N | Methyl | 3-Pyridine | 3,5-Dimethoxyphenyl | 3.99 |
| 22066 | N | H | 4-Pyridine | 3,4-Dichlorophenyl | 5.03 |
| 22032 | N | Methyl | 4-Pyridine | 3,4-Dichlorophenyl | 19.62 |
| 22012 | N | H | Phenyl | 3-Chlorophenyl | 5.67 |
| 22057 | N | Methyl | 2-Furan | 4-Methylphenyl | 5.82 |
| 22028 | N | Methyl | 3-pyridine | 4-Methoxyphenyl | 2.63 |
| 22013 | N | Methyl | Phenyl | 4-Methoxyphenyl | 5.49 |
| 22056 | N | H | 2-Furan | 4-Methylphenyl | 6.53 |
| 22052 | N | H | 3-Pyridine | 3-Chlorophenyl | 8.48 |
| 22055 | N | H | 2-Furan | Phenyl | 8.76 |
| 22051 | N | H | 3-Pyridine | Phenyl | 8.99 |
| 22009 | N | H | Phenyl | 4-Methylphenyl | 2.25 |
| 22031 | N | Methyl | 3-Pyridine | 3,4-Dichlorophenyl | 11.34 |
| 22008 | N | H | Phenyl | 4-Methoxyphenyl | 1.61 |
| 22050 | N | Methyl | 3-Pyridine | 3-Chlorophenyl | 10.62 |
| 22063 | N | Methyl | 4-Pyridine | 4-Methylphenyl | 11.64 |
| 22064 | N | Methyl | 4-Pyridine | 4-Methoxyphenyl | 11.82 |
| 22065 | N | Methyl | 4-Pyridine | Phenyl | 11.86 |
| 22058 | N | Methyl | 2-Furan | Phenyl | 12.26 |
| 22011 | N | Methyl | Phenyl | 4-Methylphenyl | 5.42 |
| 22060 | N | H | 4-Pyridine | Phenyl | 17.99 |
| 22010 | N | H | Phenyl | 3,4-Dichlorophenyl | 2.21 |
| 22049 | N | Methyl | Phenyl | 3-Chlorophenyl | 24.74 |

TABLE 2

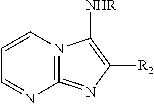

| SRI # | R | R1 | Fold left shift |
|---|---|---|---|
| 22161 | —C₆H₁₁ | 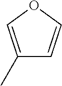 | 9.71 |
| 22162 | —C₆H₁₁ | 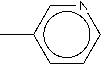 | 6.73 |
| 22156 | 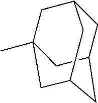 | 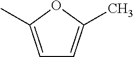 | 15.96 |
| 22160 | —C₆H₁₁ | 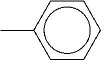 | 5.79 |
| 22154 | C₆H₁₁ | 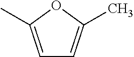 | 8.40 |
| 22158 | —CH₂C₆H₅ | 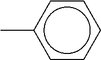 | 3.41 |
| 22159 | —CH₂C₆H₅ | 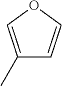 | 2.42 |
| 22155 | —CH₂CH₂CH₂CH₃ | 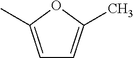 | 2.65 |

Compounds employed according to the present can be prepared by persons of ordinary skill in the art once aware of the present disclosure without undue experimentation. Accordingly, a detailed discussion of making them is not necessary. In addition, by way of example, the following example sets forth the Synthesis of SRI 22013

### Example

5,5-Dimethyl-1,3-cyclohexanedione (140 mg, 1 mmol) and p-anisidine (123 mg, 1 mmol) were refluxed in ethanol (20 ml) for 10 h. To this was added benzilidenemalononitrile (154 mg, 1 mmol) and 2 drops of TEA. The reaction mixture was refluxed for one more hour. Upon cooling a white solid precipitated out and was collected by filtration. The solid was recrystallized from ethanol to give SRI 22013 in 60% yield.

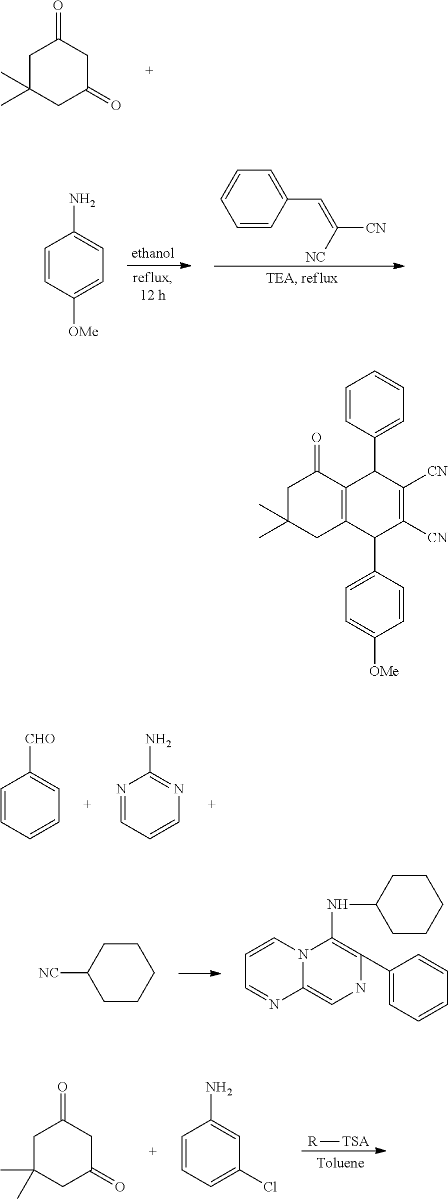

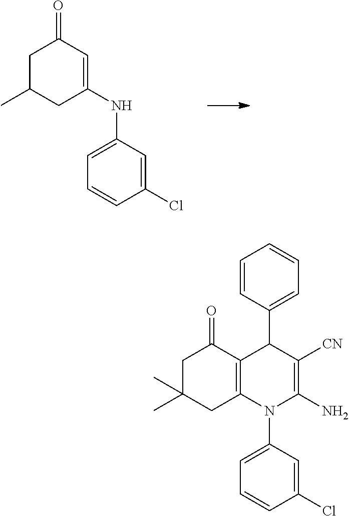

The hosts or patients treated according to this disclosure include humans and animals such as zoo or exotic animals, food animals (e.g. cattle, sheep and goats) and companion animals (e.g. dogs and cats).

### Formulations

Compounds of the present disclosure can be administered by any conventional means available for use in conjunction with pharmaceuticals. They can be administered alone, but generally administered with a pharmaceutical carrier selected on the basis of the chosen route of administration and standard pharmaceutical practice.

The pharmaceutically acceptable carriers described herein, for example, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 10 to 1000 milligrams (mg) per kilogram (kg) of body weight, with the preferred dose being 10 to about 30 mg/kg.

Dosage forms (compositions suitable for administration) typically contain from about 1 mg to about 500 mg of active ingredient per unit. In these pharmaceutical compositions, the active ingredient will ordinarily be present in an amount of about 0.5-95% weight based on the total weight of the composition.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation of a drug powder mist. Other dosage forms are potentially possible such as administration transdermally, via patch mechanism or ointment.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds of the present disclosure, alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to an animal, particularly a human, in the context of the present disclosure should be sufficient to affect a therapeutic response in the animal over a reasonable time frame. One skilled in the art will recognize that dosage will depend upon a variety of factors including a condition of the animal, the body weight of the animal, as well as the severity and stage of the condition being treated.

A suitable dose is that which will result in a concentration of the active agent in a patient which is known to affect the desired response. The preferred dosage is the amount which results in maximum inhibition of the condition being treated, without unmanageable side effects.

The size of the dose also will be determined by the route, timing and frequency of administration as well as the existence, nature, and extent of any adverse side effects that might accompany the administration of the compound and the desired physiological effect.

Useful pharmaceutical dosage forms for administration of the compounds according to the present disclosure can be illustrated as follows:

Hard Shell Capsules

A large number of unit capsules are prepared by filling standard two-piece hard gelatine capsules each with 100 mg of powdered active ingredient, 150 mg of lactose, 50 mg of cellulose and 6 mg of magnesium stearate.

Soft Gelatin Capsules

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets

A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Moreover, the compounds of the present disclosure can be administered in the form of nose drops, or metered dose and a nasal or buccal inhaler. The drug is delivered from a nasal solution as a fine mist or from a powder as an aerosol.

The term “comprising” (and its grammatical variations) as used herein is used in the inclusive sense of “having” or “including” and not in the exclusive sense of “consisting only of.” The term “consisting essentially of” as used herein is intended to refer to including that which is explicitly recited along with what does not materially affect the basic and novel characteristics of that recited or specified.

The terms “a” and “the” as used herein are understood to encompass the plural as well as the singular.

All publications, patents and patent applications cited in this specification are herein incorporated by reference, and for any and all purpose, as if each individual publication, patent or patent application were specifically and individually indicated to be incorporated by reference. In the case of inconsistencies, the present disclosure will prevail.

The foregoing description of the disclosure illustrates and describes the present disclosure. Additionally, the disclosure shows and describes only the preferred embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The embodiments described hereinabove are further intended to explain best modes known of practicing it and to enable others skilled in the art to utilize the disclosure in such, or other, embodiments and with the various modifications required by the particular applications or uses. Accordingly, the description is not intended to limit it to the form disclosed herein. Also, it is intended that the appended claims be construed to include alternative embodiments.

What is claimed is:

1. Compounds represented by the formulae: 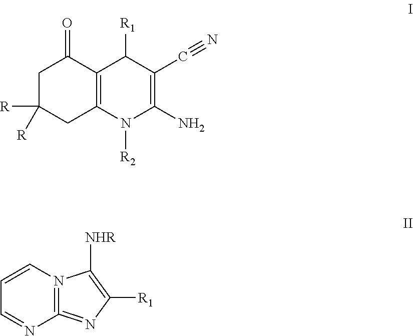
pharmaceutically acceptable salt thereof solvate thereof, and prodrug thereof;
wherein in formula I, X is N or O; each R is individually selected from the group consisting of H and alkyl; R₁ is a substituted- or unsubstituted-aryl or substituted- or unsubstituted-heteroaryl; and R₂ is phenyl or substituted phenyl; and
wherein in formula II, R is selected from the group consisting of alkyl, cycloalkyl, substituted- or unsubstituted-aryl or substituted- or unsubstituted-heteroaryl, and adamantyl; and R₂ is substituted- or unsubstituted-aryl or substituted- or unsubstituted-heteroaryl.

2. A method for enhancing the efficacy of a pharmacological agent which comprises administering to a patient in need thereof, an effective amount of a compound or pharmaceutically acceptable salt thereof, or a solvate thereof, or prodrug thereof according to claim 1.

3. The method according to claim 2 wherein said pharmacological agent is a cancer chemotherapeutic drug.

4. The method according to claim 2 wherein said pharmacological agent is a vinca alkaloid.

5. The method according to claim 2 wherein said pharmacological agent is an antifolate.

6. The method according to claim 2 wherein said pharmacological agent is a non-steroidal anti-inflammatory drug.

7. The method according to claim 2 wherein said pharmacological agent is an antibiotic.

8. The method according to claim 2 wherein said pharmacological agent is an antiparasitic agent.

9. The method according to claim 2 wherein said pharmacological agent is an antifungal drug.

10. A pharmaceutical composition comprising an effective amount of a compound or pharmaceutically acceptable salt thereof, or a solvate thereof, or prodrug thereof according to claim 1 and a pharmaceutically acceptable carrier.

11. A method for screening for compounds for use as MRP inhibitors which comprises exposing a sample compound to MRP1 over expressing human small cell tumor line and measuring the sensitivity of the cell both in the absence and the presence of a subtoxic concentration of a pharmaceutical agent.
